(19) United States
(12) Patent Application Publication
(10) Pub. No.: US 2023/0046832 A1
(43) Pub. Date: Feb. 16, 2023

(54) METHOD FOR CONTROLLING A LASER OF A TREATMENT APPARATUS, TREATMENT APPARATUS, COMPUTER PROGRAM AS WELL AS COMPUTER-READABLE MEDIUM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 17/886,968

(22) Filed: Aug. 12, 2022

(30) Foreign Application Priority Data
Aug. 16, 2021 (DE) 10 2021 121 197.2

(51) Int. Cl. A61F 9/008

(52) U.S. Cl. CPC A61F 9/00827; A61F 2009/00842; A61F 2009/00872

(57) ABSTRACT

The invention relates to a method for controlling a laser (12) of a treatment apparatus (10), comprising the steps of: generating a plurality of laser pulses (34) with a predefined energy below a photodisruption regime of a polymer material (26), irradiating the area (16) with the laser pulses (34), wherein a refractive index of the polymer material (26) changes at the irradiated irradiation point (36) depending thereon, generating a first irradiation line (38) in a first depth plane (40), wherein the first depth plane (40) is formed substantially perpendicularly to an optical axis (20) of the area (16), generating a second irradiation line (42) in a second depth plane (44) different from the first depth plane (40), wherein the first depth plane (40) and the second depth plane (44) overlap at least in certain areas viewed in the direction of the optical axis (20) and the second depth plane (44) is formed substantially perpendicularly to the optical axis (20). Further, the invention relates to a treatment apparatus (10), to a computer program, to a computer-readable medium as well as to a surgical method.

### FIELD

The invention relates to a method for controlling a laser of a treatment apparatus and to a method for performing a surgical procedure on an optical element. Further, the invention relates to a treatment apparatus, to a computer program as well as to a computer-readable medium.

### BACKGROUND

Opacities and scars within the cornea, which is also referred to as cornea, which arise by inflammations, injuries or native diseases, impair the sight. In particular in case that these pathological and/or unnaturally altered areas of the cornea are located in the axis of vision of the eye, a clear sight is considerably disturbed. Further visual disorders such as for example a reduced visual acuity or corneal curvatures can further also impair the sight. Hereto, different laser methods by means of corresponding treatment apparatuses are given from the prior art, which separate a volume body from the cornea and thus can improve the sight for a patient. Hereto, photodisruptive and ablative methods are for example known, which generate corresponding interfaces via laser pulses, and a volume body can thereby for example be removed from the cornea, whereby the injured or pathological area can be changed such that the sight is again improved.

Further, methods are already known from the prior art, in which a focused femtosecond laser with low energy and high repetition rate is employed to change a refractive index of transparent materials and tissues, such as for example a cornea, lens, contact lenses and artificial eye lenses, in non-surgical manner and thus to change the light refraction properties thereof. This method is in particular also referred to as LIRIC (Laser-Induced Refractive Index Change). In living tissues, the method does not induce wound healing or scar formation.

According to the known LIRIC methods, cylindrical or spiral paths are used to provide the phase-interleaved optical corrections. Therein, the result of the treatment is depending on the speed of the emission of the laser pulses, wherein a consistent speed is herein preset from the prior art. However, this is difficult to solve in particular in the concentric paths since in particular depending on the position of the laser pulses, the laser speed for example can no longer be kept in the center of the circular path.

### SUMMARY

Therefore, it is the object of the present invention to provide a method, a treatment apparatus, a computer program and a computer-readable medium, by means of which the disadvantages of the prior art are overcome, and in particular an improved treatment of a polymer material, in particular of a cornea, can be realized.

This object is solved by a method, a treatment apparatus, a computer program as well as a computer-readable medium according to the independent claims. Advantageous forms of configuration with convenient developments of the invention are specified in the respective dependent claims, wherein advantageous configurations of the method are to be regarded as advantageous configurations of the treatment apparatus, of the computer program and of the computer-readable medium and vice versa.

Therein, an aspect of the invention relates to a method for controlling a laser of a treatment apparatus as well as to a method for performing a surgical procedure on an optical element. A plurality of laser pulses with a predefined energy below a photodisruption regime of a polymer material of an area of an optical element is generated. Irradiating the area with the laser pulses is effected, wherein a refractive index of the polymer material changes at each irradiation point irradiated with the laser pulses depending thereon. A first irradiation line is generated within the area by means of a plurality of irradiation points in a first depth plane of the optical element, wherein the first depth plane is formed substantially perpendicularly to an optical axis of the area. Generating a second irradiation line within the area with a plurality of irradiation points in a second depth plane of the optical element different from the first depth plane is effected, wherein the first depth plane and the second depth plane overlap at least in certain areas viewed in the direction of the optical axis and the second depth plane is formed substantially perpendicularly to the optical axis of the area.

Thus, it is in particular proposed that the first depth plane and the second depth plane are formed substantially parallel to each other, in particular parallel to each other. Thus, the two generated irradiation lines in particular also overlap at least in certain areas viewed in the direction of the optical axis.

Presently, the irradiation lines are in particular substantially straight irradiation lines. The irradiation lines can be particularly simply generated since the speed does not have to change in particular for example in the direction of a center of the area, which is to be treated, to still reliably perform a treatment. Therein, it is in particular provided that an astigmatic correction can be realized based on an irradiation line. In particular by the second irradiation line, which is for example located higher within the optical element viewed in the optical axis, a spherical correction can then in turn be realized by the overlap of the two irradiation lines.

Thus, it is allowed in simple manner that the corresponding correction can be performed by the two irradiation lines without a laser pulse speed having to be changed. Thereby, the polymer material can be corrected in simplified manner.

The polymer material is in particular a human or animal cornea and/or a lens of an eye. However, it is to be further also mentioned at this place that the method can also be applied in artificial contact lenses or artificial intraocular lenses. Thus, the polymer material is in particular for example a biopolymer material, which is synthesized in a cell of a living being, for example in the form of polysaccharides, proteins, nucleic acids or the like. However, artificial polymer materials such as for example collagen can further also be correspondingly changed with the method according to the invention. Then, the cornea or the lens of the eye can for example be considered as biopolymer material, while for example contact lenses or intraocular lenses can be regarded as collagen.

For example, the optical element can then be an eye or a vitreous body. The area in turn describes that area within the polymer material, which for example encompasses the pathological area or area to be treated.

Thus, the laser pulses are in particular generated with the predefined energy such that they are generated below the photodisruption regime, which means that photodisruption bubbles are not generated within the cornea, whereby only the refractive index is changed within the irradiation points, whereby light beams are in turn refracted in the area after the treatment different than before, which results in a correction on the optical element.

According to an advantageous form of configuration, the second irradiation line in the second depth plane is generated higher than the first irradiation line viewed in relation to the optical axis. In other words, it can be provided that the first irradiation line is generated deeper, for example deeper in the cornea, than the second irradiation line. This in particular has the advantage, for example if the irradiation line located deeper viewed in the direction of the optical axis should be first generated and thereafter the irradiation line located higher should be generated, thus, mutual impairments in generating the irradiation lines cannot be registered. Thus, the first irradiation line and the second irradiation line can be reliably generated without mutual impairment.

It is further advantageous if a plurality of substantially parallel first irradiation lines is generated in the area at least in the first depth plane and/or a plurality of substantially parallel second irradiation lines is generated in the area at least in the second depth plane. In particular, parallel can be understood by “substantially parallel”. Thus, a grid structure can in particular be provided in the area of the overlap of the first depth plane or second depth plane, which in turn can be used for correcting a visual impairment.

It is further advantageous if the respective plurality of irradiation lines is generated in the first depth plane and in the second depth plane such that they form a grid structure viewed in the direction of the optical axis. Thus, a spherical correction can in particular be very reliably realized within the overlap area based on the generated grid structure.

In a further advantageous form of configuration, a third irradiation line is generated within the area in a third depth plane of the optical element different from the first depth plane and from the second depth plane, wherein the first depth plane, the second depth plane and the third depth plane overlap at least in certain areas and the third depth plane is formed substantially perpendicularly to the optical axis of the area. Thereby, a sphero-cylindrical correction can be generated from the three irradiation lines. In particular, it can thus for example be realized that the treatment apparatus has to generate the irradiation lines only in four different orientations, whereby the treatment apparatus can be extremely simply formed. In particular, these orientations can for example only relate to 0°, 45°, 90° and 135°. Depending on the correction to be generated, it can then in turn be decided, which three irradiation lines and at which angles to each other, respectively, these irradiation lines have to be formed. For example, this can be decided depending on a correction value for the eye, which is for example stored in a control device within the treatment apparatus, by the control device.

In a further advantageous form of configuration, at least the second irradiation line is generated such that it has a first relative non-zero angle to the first irradiation line. In particular by the relative non-zero angle, thus, different corrections can be realized. In particular, it can be decided depending on the correction value, at which relative angle the first irradiation line has to be formed to the second irradiation line to be able to perform a corresponding correction, for example on the eye.

It has further proven advantageous if at least the second irradiation line is generated such that it has a first relative angle of substantially 90° or substantially 45°. In particular with for example an orientation of 90°, a spherical correction can be realized. With the orientation of for example 45° and a different change of the refractive index in the first irradiation line and in the second irradiation line, different sphero-cylindrical corrections can be realized.

Further, it has proven advantageous if the first relative angle is generated depending on patient information. For example, the patient information can comprise corresponding correction values, which are required to correct a visual disorder. Depending thereon, the control device of the treatment apparatus can then in turn be formed to determine the corresponding intensities and relative angles in autonomous and automated manner.

It has further proven advantageous if the laser pulses for the first depth plane are generated with a first preset energy and the laser pulses for the second depth plane are generated with a second preset energy different from the first preset energy. Thus, spherical corrections or sphero-cylindrical corrections can be performed already with little effort, only with different energies within the different depth planes. This allows a simple treatment and a less complexly structured treatment apparatus.

According to a further advantageous form of configuration, a third irradiation line is generated within the area in a third depth plane of the optical element different from the first depth plane and the second depth plane, wherein the first depth plane, the second depth plane and the third depth plane overlap at least in certain areas and the third depth plane is formed substantially perpendicularly to the optical axis of the area, wherein a first relative angle is formed between the first irradiation line and the second irradiation line and a second relative angle, which is different from the first relative angle, is formed between the first irradiation line and the third irradiation line. For example, the first depth plane can have an angle of 0°, and an angle of 90° can be formed between the first depth plane and the second depth plane. Then, a relative angle of 45° or 135° can be formed between the first depth plane and the third depth plane. Thus, sphero-cylindrical corrections can be realized by means of simple shifts of the angles of the three depth planes or the three irradiation lines to each other.

It has further proven advantageous if the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz. Thereby, the laser pulses can in particular be generated below the photodisruption regime, which only results in a change of the refractive index. Thereby, the method and in particular the change of the refractive index can be reliably performed without performing an invasive intervention in the cornea.

In a further advantageous form of configuration, topographic and/or pachymetric and/or morphologic data of the optical element, in particular of the eye, in particular of the cornea and/or the lens, is taken into account in controlling the laser. In particular, this data can for example be determined already before a treatment. Based on this data, the treatment can then be reliably performed.

A second aspect of the invention relates to a treatment apparatus with at least one eye surgical laser and with at least one control device for the laser or lasers, which is formed to perform the steps of the method according to the first aspect.

Preferably, the treatment apparatus is formed as a rotation scanner and for example comprises a beam deflection device hereto.

In an advantageous form of configuration of the treatment apparatus, the treatment apparatus comprises a storage device for at least temporary storage of at least one control dataset, wherein the control dataset or datasets include(s) control data of individual laser pulses on or in the optical element, and includes at least one beam deflection device for beam guidance and/or beam shaping and/or beam deflection and/or beam focusing of a laser beam of the laser. Therein, the mentioned control datasets are usually generated based on a measured topography and/or pachymetry and/or morphology of the optical element to be treated, in particular of the cornea or lens to be treated of the pathologically and/or unnaturally altered area within the optical element.

Therein, it can be provided that the treatment apparatus comprises a single storage device and a single control device. Alternatively, it can be provided that different storage devices and control devices are formed within the treatment apparatus to perform a corresponding control of the laser.

Further features and the advantages thereof can be taken from the descriptions of the first inventive aspect, wherein advantageous configurations of the first inventive aspect are to be regarded as advantageous configurations of the respectively other inventive aspect.

A third aspect relates to a computer program including commands, which cause the treatment apparatus according to the second inventive aspect to execute the method steps according to the first inventive aspect. A fourth aspect of the invention relates to a computer-readable medium, on which the computer program according to the third inventive aspect is stored.

Further features and the advantages thereof can be taken from the descriptions of the first and second inventive aspects, wherein advantageous configurations of each inventive aspect are to be regarded as advantageous configurations of the respectively other inventive aspect.

### BRIEF DESCRIPTION OF DRAWINGS

Further features are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 shows a schematic view of an embodiment of a treatment apparatus.

FIG. 2 shows a schematic perspective view of an optical element.

FIG. 3 shows a schematic top view to irradiation lines of a first embodiment according to the method.

FIG. 4 shows a further schematic top view to irradiation lines of a second embodiment of the method.

FIG. 5 shows a third schematic top view to irradiation lines of a further, third embodiment of the method.

In the figures, identical or functionally identical elements are provided with identical reference characters.

### DETAILED DESCRIPTION

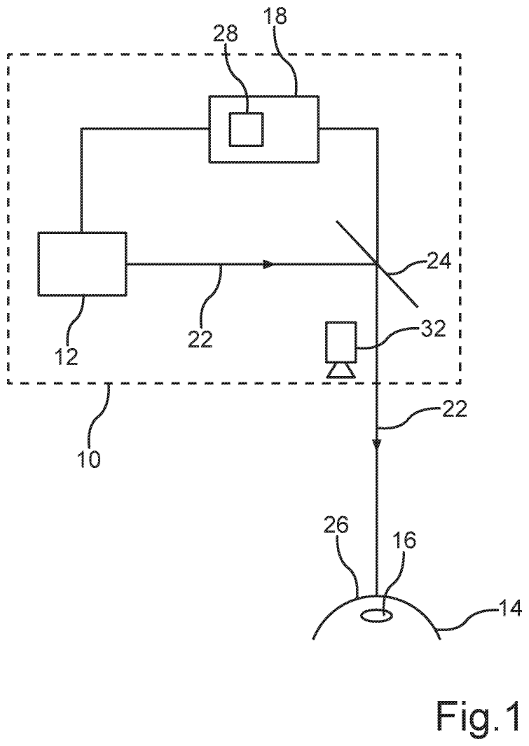

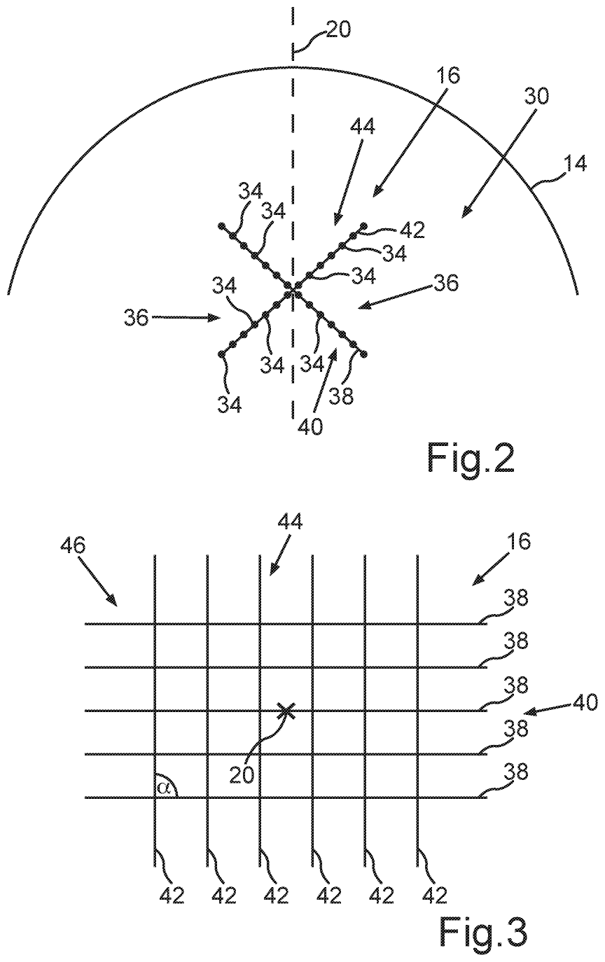

FIG. 1 shows a schematic representation of a treatment apparatus 10 with an eye surgical laser 12 for the treatment of a patient, in particular for the treatment of an eye 14 of a patient, wherein the eye 14 is also referred to as optical element 14 below. One recognizes that a control device 18 for the laser 12 is formed besides the laser 12. This form of configuration with a control device 18 is to be purely exemplarily regarded. It can be provided that the treatment apparatus 10 also comprises a plurality, in particular more than two, of control devices 18. For example, the control device 18 can emit pulsed laser pulses 34 (see FIG. 2) in a predefined pattern into the eye 14, for example into an area 16, wherein the position of the area 16 is selected in this embodiment such that a pathological and/or unnaturally altered area within a stroma of the eye 14 is enclosed.

Furthermore, one recognizes that the laser beam 22 generated by the laser 12 is deflected towards the eye 14 by means of a beam deflection device 24 such as for example a scanner, in particular a so-called rotation scanner. The beam deflection device 24 is also controlled by the control device 18 to for example generate irradiation lines 38, 42, 48 (see FIG. 3). For example, the beam deflection device 24 can comprise one or else two mirrors, which are formed for deflecting the impinging laser beam 22.

In the present embodiment, the illustrated laser 12 is a laser 12, which emits the laser pulses 34 in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz. Thereby, the laser pulses 34 can in particular be generated below the photodisruption regime, which results only in a change of the refractive index. Thereby, the method and in particular the change of the refractive index can be reliably performed without performing an invasive intervention for example in a cornea 30 (see FIG. 2). Further, the laser beam 22 can be generated both as a working beam with a lower energy than a treatment beam, but also as a treatment beam itself.

In addition, the control device 18 comprises a storage device 28 for at least temporary storage of at least one control dataset, wherein the control dataset or datasets include(s) control data for positioning and/or for focusing individual laser pulses 34 in or on the eye 14. The position data and/or focusing data of the individual laser pulses 34 are generated based on a previously measured topography and/or pachymetry and/or the morphology of the eye 14 and the for example pathological and/or unnaturally altered area within the stroma of the eye 14.

FIG. 2 shows a schematic top view to an embodiment of a polymer material 26, which presently in particular corresponds to the eye 14 and below to the optical element 14. In particular, the eye 14 in turn for example comprises a cornea 30, in which the area 16 is formed, which is to be corrected.

The polymer material 26 is preferably a human or animal cornea 30. However, non-biopolymers such as for example contact lenses or intraocular lenses can also be correspondingly processed. Therein, the control of the laser 12 is effected such that topographic and/or pachymetric and/or morphologic data, in particular of the cornea 30 as the polymer material 26, is taken into account.

In the method for controlling the laser 12 of the treatment apparatus 10, a plurality of laser pulses 34 with a predefined energy below a photodisruption regime of the polymer material 26 of the area 16 of the optical element 14 is generated. Irradiating the area 16 with the laser pulses 34 is effected, wherein a refractive index of the polymer material 26 changes at each of irradiation points 36 irradiated with the laser pulses 34 depending thereon. A first irradiation line 38 is generated within the area 16 by means of the plurality of irradiation points 36 in a first depth plane 40 of the optical element 14, wherein the first depth plane 40 is formed substantially perpendicularly to the optical axis 20 of the area 16. Generating a second irradiation line 42 within the area 16 with a plurality of irradiation points 36 in a second depth plane 44 of the optical element 14 different from the first depth plane 40 is effected, wherein the first depth plane 40 and the second depth plane 44 overlap at least in certain areas viewed in the direction of the optical axis 20 and the second depth plane 44 is formed substantially perpendicularly to the optical axis 20 of the area 16.

In particular, FIG. 2 shows that the first depth plane 40 and the second depth plane 44 are thus formed substantially parallel to each other. Therein, it can in particular be provided that the second irradiation line 42 in the second depth plane 44 is generated higher than the first irradiation line 38 viewed in relation to the optical axis 20. In other words, the first irradiation line 38 can for example be generated in a deeper plane of the cornea 30 than the second irradiation line 42.

FIG. 3 shows a first embodiment according to the method. In particular, FIG. 3 shows that a plurality of substantially parallel irradiation lines, in particular first irradiation lines 38, is generated at least in the first depth plane 40 and/or a plurality of substantially parallel irradiation lines, in particular second irradiation lines 42, is generated in the area 16 at least in the second depth plane 44. FIG. 3 in particular shows that the respective plurality of irradiation lines 38, 42 is generated in the first depth plane 40 and in the second depth plane 44 such that they form a grid structure 46 viewed in the direction of the optical axis 20.

In the present embodiment, it is in particular shown that each irradiation line 38, 42 is a substantially straight irradiation line in particular extending parallel to an adjacent irradiation line, which are adequately separated from each other, in particular formed parallel to each other, whereby an astigmatism can in particular be corrected. In particular with the two irradiation lines 38, 42, which have the same “power”, thus have been generated with the same power of the laser pulses 34 or have been generated with the same energy, but are formed at an angle of 90° to each other, a spherical correction can be generated within the overlap area. Namely, FIG. 3 in particular also shows that at least the second irradiation line 42 has a relative non-zero angle a to the first irradiation line 38. Presently, the second irradiation line 42 in particular has an angle of 90°.

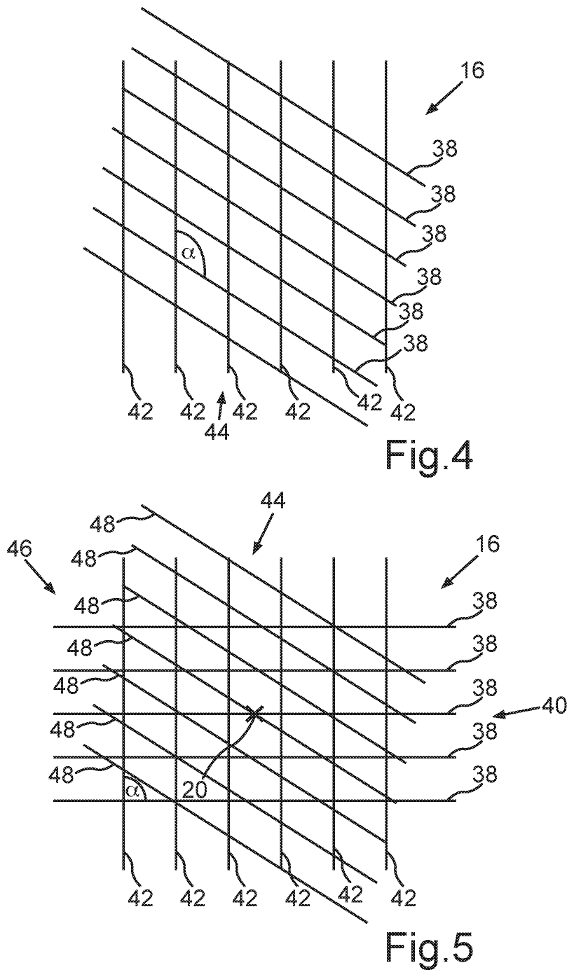

FIG. 4 shows a further schematic embodiment according to an embodiment of the method. Presently, the two irradiation lines 38, 42 are again shown, which are formed in the first depth plane 40 and in the second depth plane 44. Presently, a relative angle a of for example 135° or 45° is in particular formed between the first irradiation line 38 and the second irradiation line 42. In particular, the relative angle a is generated depending on patient information. Further, it can also be provided that the laser pulses 34 for the first depth plane 40 are generated with a first preset energy and the laser pulses 34 for the second depth plane 44 are generated with a second preset energy different from the first preset energy. Thereby, it is allowed that they also, as presently shown with an angle lower than 90°, result in a sphero-cylindrical correction in that they generate different refractive indices. Thereby, it is allowed that a plurality of sphero-cylindrical corrections can already be allowed by means of the two irradiation lines 38, 42 and the different preset energies.

FIG. 5 shows a third embodiment according to the method. Presently, it is in particular shown that a third irradiation line 48 is generated within the area 16 in a third depth plane 50 of the optical element 14 different from the first depth plane 40 and from the second depth plane 44, wherein the first depth plane 40, the second depth plane 44 and a third depth plane 50 are formed substantially perpendicularly to the optical axis 20 of the area 16. In particular, they overlap, as presently shown in FIG. 5. In particular, it is presently also shown that a first relative angle a is formed between the first irradiation line 38 and the second irradiation line 42 and a second relative angle β, which is different from the first relative angle α, is formed between the first irradiation line 38 and the third irradiation line 48. Presently, it is in particular shown that the first relative angle α can for example have an angle of 90° and the second relative angle β can have an angle of 135° or 45°. By the three irradiation lines 38, 42, 48, a plurality of sphero-cylindrical corrections can be realized based on the different orientation of the irradiation lines 38, 42, 48 to each other. In particular, only four orientations, namely 0°, 45°, 90° and 135°, of the irradiation lines 38, 42, 48 are thus required to perform a plurality of sphero-cylindrical corrections. Thereby, the treatment apparatus 10 can be substantially very simply formed since only the irradiation lines 38, 42, 48 have to be generated at the respective angles α, β.

Further, it is in particular provided that at least the number of the irradiation lines 38, 42, 48 and/or the first relative angle a and/or the second relative angle β are generated depending on patient information.

1. A method for controlling a laser of a treatment apparatus, comprising the steps of:
generating a plurality of laser pulses with a predefined energy below a photodisruption regime of a polymer material of an area of an optical element;
irradiating the area with the laser pulses, wherein a refractive index of the polymer material changes at each irradiation point irradiated with the laser pulses depending thereon;
generating a first irradiation line within the area by means of a plurality of irradiation points in a first depth plane of the optical element, wherein the first depth plane is formed substantially perpendicularly to an optical axis of the area;
generating a second irradiation line within the area with a plurality of irradiation points in a second depth plane of the optical element different from the first depth plane, wherein the first depth plane and the second depth plane overlap at least in certain areas viewed in the direction of the optical axis and the second depth plane is formed substantially perpendicularly to the optical axis of the area.

2. The method according to claim 1, charactcrizcd in that wherein the second irradiation line in the second depth plane is generated higher than the first irradiation line viewed in relation to the optical axis.

3. The method according to claim 1, charactcrizcd in that wherein a plurality of substantially parallel first irradiation lines is generated in the area at least in the first depth plane and/or a plurality of substantially parallel second irradiation lines is generated in the area at least in the second depth plane.

4. The method according to claim 3, wherein the respective plurality of irradiation lines in the first depth plane and in the second depth plane is generated such that they form a grid structure viewed in the direction of the optical axis.

5. The method according to claim 1, wherein a third irradiation line is generated within the area in a third depth plane of the optical element different from the first depth plane and from the second depth plane, wherein the first depth plane, the second depth plane and the third depth plane overlap at least in certain areas and the third depth plane is formed substantially perpendicularly to the optical axis of the area.

6. The method according to claim 1, charactcrizcd in that wherein at least the second irradiation line is generated such that it has a first relative non zero angle to the first irradiation line.

7. The method according to claim 6, charactcrizcd in that wherein at least the second irradiation line is generated such that it has a the first relative angle is substantially 90° or substantially 45° degrees.

8. The method according to claim 6, wherein at least the first relative angle is generated depending on patient information.

9. The method according to claim 1, wherein the laser pulses for the first depth plane are generated with a first preset energy and the laser pulses for the second depth plane are generated with a second preset energy different from the first preset energy.

10. The method according to claim 1, wherein a third irradiation line is generated within the area in a third depth plane of the optical element different from the first depth plane and from the second depth plane, wherein the first depth plane, the second depth plane and the third depth plane overlap at least in certain areas and the third depth plane is formed substantially perpendicularly to the optical axis of the area, wherein a first relative angle is formed between the first irradiation line and the second irradiation line and a second relative angle, which is different from the first relative angle, is formed between the first irradiation line and the third irradiation line.

11. The method according to claim 1, wherein the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

12. The method according to claim 1, wherein the control of the laser is effected such that topographic and/or pachymetric and/or morphologic data of a cornea as the polymer material is taken into account.

13. A treatment apparatus with at least one eye surgical laser and with at least one control device for the laser or lasers, which is formed to execute the steps of the method according to claim 1.

14. The treatment apparatus according to claim 13, characterized in that wherein the control device:
comprises at least one storage device for at least temporary storage of at least one control dataset, wherein the control dataset or datasets include(s) control data for positioning and/or for focusing individual laser pulses in the polymer material; and
includes at least one beam device for beam guidance and/or beam shaping and/or beam deflection and/or beam focusing of a laser beam of the laser.

15. A computer program including commands, which cause a treatment apparatus with at least one eye surgical laser and with at least one control device for the laser or lasers to execute the method steps according to claim 1.

16. A non-transitory computer-readable medium, on which the computer program according to claim 15 is stored.

17. A method for performing a surgical procedure on an optical element of a human or animal, comprising the steps of:
generating a plurality of laser pulses with a predefined energy below a photodisruption regime of a polymer material of an area of an optical element;
irradiating the area with the laser pulses, wherein a refractive index of the polymer material changes at each irradiation point irradiated with the laser pulses depending thereon;
generating a first irradiation line within the area by means of a plurality of irradiation points in a first depth plane of the optical element, wherein the first depth plane is formed substantially perpendicularly to an optical axis of the area;
generating a second irradiation line within the area with a plurality of irradiation points in a second depth plane of the optical element different from the first depth plane, wherein the first depth plane and the second depth plane overlap at least in certain areas viewed in the direction of the optical axis and the second depth plane is formed substantially perpendicularly to the optical axis of the area.

18. The method according to claim 17, wherein the second irradiation line in the second depth plane is generated higher than the first irradiation line viewed in relation to the optical axis.

19. The method according to claim 17, wherein a plurality of substantially parallel first irradiation lines is generated in the area at least in the first depth plane and/or a plurality of substantially parallel second irradiation lines is generated in the area at least in the second depth plane.

20. The method according to claim 19, wherein the respective plurality of irradiation lines in the first depth plane and in the second depth plane is generated such that they form a grid structure viewed in the direction of the optical axis.

21. The method according to claim 17, wherein a third irradiation line is generated within the area in a third depth plane of the optical element different from the first depth plane and from the second depth plane, wherein the first depth plane, the second depth plane and the third depth plane overlap at least in certain areas and the third depth plane is formed substantially perpendicularly to the optical axis of the area.

22. The method according to claim 17, wherein at least the second irradiation line is generated such that it has a first relative angle to the first irradiation line.

23. The method according to claim 22, charactcrizcd in that wherein at least the second irradiation line is generated such that the first relative angle is of substantially 90° or substantially 45° degrees.

24. The method according to claim 22, wherein at least the first relative angle is generated depending on patient information.

25. The method according to claim 17, in that wherein the laser pulses for the first depth plane are generated with a first preset energy and the laser pulses for the second depth plane are generated with a second preset energy different from the first preset energy.

26. The method according to claim 17, wherein a third irradiation line is generated within the area in a third depth plane of the optical element different from the first depth plane and from the second depth plane, wherein the first depth plane, the second depth plane and the third depth plane overlap at least in certain areas and the third depth plane is formed substantially perpendicularly to the optical axis of the area, wherein a first relative angle is formed between the first irradiation line and the second irradiation line and a second relative angle, which is different from the first relative angle, is formed between the first irradiation line and the third irradiation line.

27. The method according to claim 17, wherein the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

28. The method according to claim 17, wherein the control of a laser is effected such that topographic and/or pachymetric and/or morphologic data of a cornea as the polymer material is taken into account.
